(19) United States
(12) Patent Application Publication
(10) Pub. No.: US 2002/0138004 A1
(43) Pub. Date: Sep. 26, 2002

(54) Ultrasound imaging method using redundant synthetic aperture concepts

(76) Inventors: Fred M. Dickey, Albuquerque, NM (US); Armin W. Doerry, Albuquerque, NM (US); Alan K. Morimoto, Nashville, TN (US)

Correspondence Address: Paul A. Gottlieb, A.G.C., GC-62 (FORSTL) MS -6F-067, United States Department of Energy, 1000 Independence Avenue, S.W., Washington, DC, 20585, US

(21) Appl. No.: 09/814,103

(22) Filed: Mar. 22, 2001

(51) Int. Cl. A61B 8/00

(52) U.S. Cl. 600/443

(57) ABSTRACT

A method for improving the resolution of ultrasound images using synthetic aperture concepts. A two-dimensional image is obtained using ultrasound imaging apparatus located at a first position outside the target area and at a predetermined distance from a center point within the target area. The imaging apparatus is then moved along an arc to a new position and another two-dimensional ultrasound image is obtained. This process is repeated through up to a full 360° around the target area. Image data for each pixel is combined, calculated and transformed for all image positions to produce a highly resolved image of that pixel. The process is repeated for each pixel until all the pixels within the target area are fully resolved, thereby producing a complete, highly resolved over-all image.

### CONTRACTUAL ORIGIN OF THE INVENTION

The United States Government has rights in this invention under a contract number DE-AC04-94AL85000 with the Department of Energy.

### BACKGROUND OF THE INVENTION

1. Field of the Invention

The present invention relates to ultrasound imaging. More particularly, the invention relates to improved ultrasound imaging using redundant imaging and synthetic aperture concepts.

2. Related Art

There is much interest in improved imaging of fine internal details in a host of technological fields. This is particularly true in the field of medicine. The internal imaging methods used in the medical fields—such as x-ray, CAT scan (an improved x-ray technique), and ultrasound—are capable of providing images of internal organs and have allowed medical professionals to detect, in a non-invasive manner, many afflictions for treatment.

Ultrasound, one of the most common of these imaging methods, is frequently used in prenatal care because of its safety. Ultrasound allows doctors and other medical professionals to check on the general health of a baby by safely providing an image of the baby before birth, thus enabling early detection of many potential problems.

Ultrasound methods obtain images of an object by using high frequency sound waves. The sound waves are sent from a transducer to the object to be examined and are reflected by the object back to the transducer. The pattern in which the sound waves “bounce back” to the transducer can be used to calculate a two-dimensional image. The limited resolution of the images obtained by traditional ultrasound machines is, however, such that the images are not useful for certain medical purposes. While a single ultrasound machine is useful in providing a general two-dimensional image, the resolution provided is such that the machine cannot be used for certain delicate applications, e.g., the detection of breast cancer.

To date, other imaging technologies, such as standard x-ray and CAT scan, have been used to produce the higher resolution images necessary for breast cancer detection and for other medical purposes requiring high-resolution imaging. However, x-rays can be cancer causing in and of themselves. The danger is minimal for an individual x-ray procedure, but x-rays suffer from the disadvantage that there is only a limited number of times per day that x-rays can be used before there is an unacceptable risk to the patient. CAT scans, in addition to suffering from the same limitations as standard x-ray techniques, are extremely expensive and have limited applications.

One alternative approach for creating high-resolution images would be to surround a patient with ultrasound machines. A large number of ultrasound machines providing images of the same two-dimensional area from a multitude of angles would increase the resolution of each pixel within that two-dimensional area to a level where very fine or small abnormalities, such as breast cancer, could be detected. However, such an apparatus would suffer from some limitations similar to those of a CAT scan. Providing a ring of ultrasound machines surrounding a patient would obviously be bulky and prohibitively expensive.

As such, there remains a need for an inexpensive method to generate high-resolution ultrasound images.

### SUMMARY OF THE INVENTION

In accordance with the invention, a method is provided for improving the resolution of images produced by ultrasound machines, wherein an ultrasound image is obtained of a target area using an ultrasound imaging apparatus located at a first position outside the target area and at a predetermined distance from a center point within the target area. The ultrasound imaging apparatus is moved along an arc spaced from the center point of the target area to a new position and a further ultrasound image of the target area is obtained. This process is repeated for a plurality of further positions along the arc, as desired. The phase of sound waves returning from a pixel within the target area to the ultrasound apparatus is calculated for all the imaging positions until complete image data for the pixel is obtained. The image data for the pixel are combined and calculated for all of the positions of the apparatus to produce a higher resolution image of the pixel. This is repeated for each pixel within the target area.

Preferably, the ultrasound transducer would be moved about 2-5° around the arc between positions, but the imaging method of this invention will function with any angle of change between individual imaging positions.

Advantageously, the method can be used whether the ultrasound images are coherent images or incoherent images. If the ultrasound images are incoherent images, they are combined, using a synthetic aperture algorithm, into a much higher resolution set of incoherent images.

Advantageously, the method will be used in the medical field and, more particularly, in the detection of cancer.

More specifically, the method can be used for the safe, effective detection of breast cancer.

Other features of this invention will be set forth in, or will be apparent from, the detailed description of the embodiments that follow.

### BRIEF DESCRIPTION OF THE DRAWINGS

FIG. 1 is a schematic diagram showing a single ultrasound apparatus in use about a center point.

FIG. 2 is a schematic diagram showing the mathematical extrapolation and resolution of a single pixel.

FIG. 3 is a flow diagram of the imaging process as shown in FIGS. 1 and 2, as applied to ultrasound machines that store coherent images.

FIG. 4 is a flow diagram of the imaging process as shown in FIGS. 1 and 2, as applied to ultrasound machines that do not store coherent images.

### DESCRIPTION OF THE PREFERRED EMBODIMENTS

As indicated above, according to the present invention, an inexpensive method is provided for obtaining highly resolved ultrasound images. In general, the invention uses a synthetic aperture concept to simulate the resolution that would be provided by using multiple surrounding ultrasound devices. Synthetic receiver aperture imaging is discussed, e.g., in Nock et al., “Synthetic Receive Aperture Imaging with Phase Connection for Motion and for Tissue Inhomogneities—Part I; Basic Principles,” IEEE Transactions on Ultrasonics, Ferroelectrics, and Frequency Control, Vol. 39, No. 4, 489-495, July 1992; and Trahey et al., “Synthetic Receive Aperture Imaging for Motion and for Tissue Inhomogneities—Part II: Effects of and Connection for Motion,” IEEE Transactions on Ultrasonics, Ferroelectrics, and Frequency Control, Vol. 39, No. 4, 496-501, July 1992. The term synthetic aperture concept refers, in brief, to the simulation of the use of multiple devices, so as to obtain the superior resolution provided thereby, by the repeated use of a single mobile imaging apparatus, together with a mathematical combining of the images from the single mobile imaging apparatus to produce a highly resolved image.

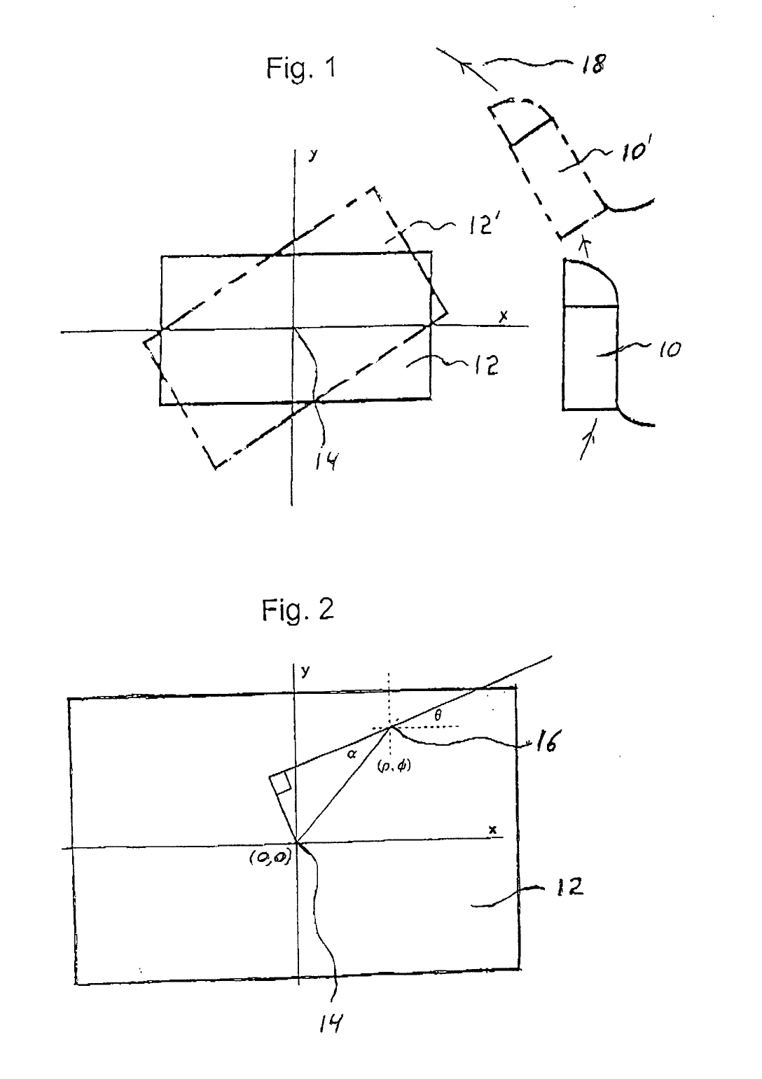

Referring to FIG. 1, there is shown a movable ultrasound transducer 10 positioned directly above a target area 12. Within this target area 12 is a center point 14. The ultrasound transducer 10 captures a two-dimensional ultrasound image of the target area 12 and the image is recorded. This two-dimensional to ultrasound image captured by transducer 10 in the position thereof shown in solid lines is of a low definition because the sound waves returning to the transducer 10 are from one angle only. To increase the number of angles of surveillance and, hence, improve the resolution, the transducer 10 is moved 2-5° along a circular arc 18 to a new position indicated at 10′. The transducer 10′ captures a second two-dimensional image of target area 12′ from the new angle corresponding to its position in the circular arc 18. The sound waves return to the transducer 10 and the second image is recorded. The resolution is increased because sound waves from these two angles provide a more complete picture than sound waves from the first, single angle shown in solid lines in FIG. 1.

To continue the process, the transducer 10 is moved a further 2-5° about the circular arc 18 and the imaging process is repeated. The overall process can be repeated until the transducer 10 has moved up to a full 360° through the circular arc 18.

It will be appreciated that the transducer 10 moves in the circular arc 18 around, and at a constant distance from, the center point 14. Referring to FIG. 2, the center point 14 is positioned at the intersection of a pair of X and Y coordinates, this intersection being denoted in FIG. 2 as (0, 0). The transducer 10 moves within the XY plane and relative to the X and Y coordinates of the plane. In this embodiment, the ultrasound transducer 10 is always the same distance from the center point 14 as it moves around arc. This arc may be up to 360°. Residual body motion and movement that would otherwise diminish resolution quality can be mitigated using autofocus techniques.

After all the multiple images are captured and recorded, a redundant imaging method is employed to improve image definition. The images taken about the arc 18 are combined to form a composite image. The images can be is combined to form a composite image whether the images are coherent (i.e., both magnitude and phase information are recorded or incoherent (i.e., magnitude information only is recorded). Commonly, the redundant images are incoherent and are thus combined as a weighted average of the magnitudes only. The imaging system processes the combined image at each pixel and calculates the ideal resolution obtainable. The image of the representative pixel 16 within the target area 12 will be in the form of a set of plane waves returned to the transducer 10 from the representative pixel 16. The phase of the returning waves is determined by the location of the representative pixel 16 relative to the location of the transducer 10. The combination of sound waves from the representative pixel 16 returned to the transducer 10 for various positions of the transducer 10 around the arc 18, and the varying phases of the sound waves which are returned, when processed in accordance with this invention provide a complete, high resolution, final image of the representative pixel 16.

Improved resolution can also be obtained if coherent images are combined, that is, if the waves from the separate images are in phase with each other. Coherent image addition requires the use of a coherent imaging system. Coherent imaging systems are linear and preserve both signal amplitude and phase. The General Electric LOGIQ 700 ultrasound system provides this capability. To achieve improved resolution, each coherent image is processed by adding a phase term prior to combination.

As shown in FIG. 2, the representative pixel 16 is located in the target area 12 with respect to the center of rotation 14 at coordinates (ρφ). A representative plane wave will return from this pixel back to the transducer 10. The position of the pixel 16 relative to the transducer 10 is a function of the phase of the wave upon return.

Various phases of waves from the representative pixel 16, resulting from the different positions of the transducer 10 are calculated, and multiplied by a phase factor. This is repeated for each pixel in the sub-images until a more clearly resolved picture of all of the pixels appears.

The phase of the scattered wave relative to the center of rotation, and as a function of the transducer angle data, is given by:

s(x,y)=e^{-ik(x cos θ+y sin θ)}e^{i2kΔ}. Eq. (1)

Where 2Δ is determined by noting that

α=π/4−(π/4)−φ+θ=θ−φ.

Giving

Δ=ρ cos (φ−θ). Eq. (2)

Eq. (1) then becomes

s(x,y)=e^{-ik(x cos θ+y sin θ-2ρ cos (φ-θ)).} Eq. (3)

The field at (r, β) with respect to the point scattered is obtained by substituting,

x=ρ cos φ+r cos β,

y=ρ cos φ+r sin β.

This gives,

s(r, β)=e^{-ik(β cos φ cos θ+r cos β cos θ+ρ sin φ sin θ+r sin β sin θ)}e^{-i2kρ cos (φ-θ)} =e^{ikρ cos (φ-θ)}e^{-ikr cos (β-θ).} Eq. (4)

For each (discrete θ) θₙ one obtains a scattered field of the form

sₙ(r, β)=e^{-ikρ cos (φ-θn)}e^{-ikr cos (β-θn).} Eq. (5)

Each image can be multiplied by

e^{-ikρ cos (φ-θn)}

and the total phases of the resulting images are summed to give

|(r, β)=Σe^{-ikr cos (β-θn).} Eq. (6)

In the continuous case the summation becomes an integral resulting in an image of the form:

|(r, β)=0∫^{2π}e^{-ikr cos (β-θ)}dθ. Eq. (7)

This result defines the limiting resolution for such systems.

It is noted that Eq. (1) and subsequent equations should be multiplied by an amplitude function (δfunction), and, if this is done, the resulting image is a convolution of the above impulse response with the input. Further, this analysis can be extended to include diffraction and system image impulse response.

For coherent processing it is more convenient to describe the algorithm in rectangular coordinates using discrete pixels as variables. If we let σ cos θ=xₒ and σ sin φ=yₒ, Eq. (3) can be written in the form

s(x, y)=e^{-ik((Xi-Xo) cos θ+(Yj-Yo) sin θ)}e^{ik(Xo cos θ+Yo sin θ)} Eq. (8)

where the subscripts (i, j) denote discrete pixel locations (not shown).

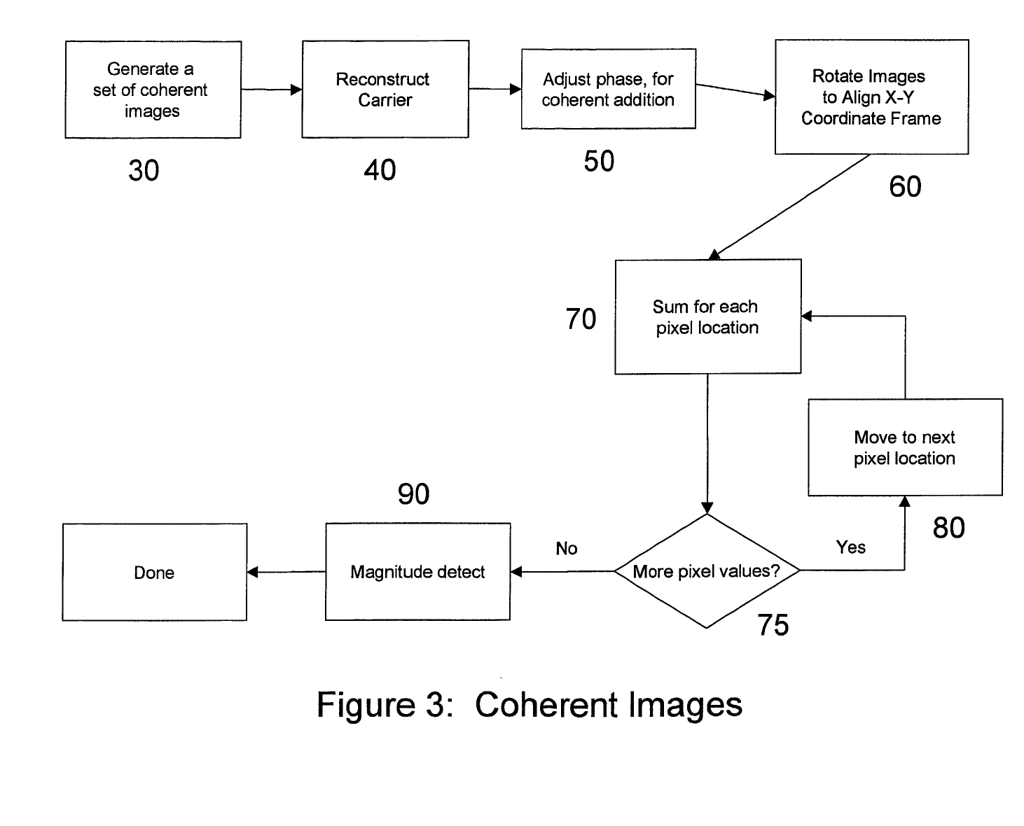

Based on Equation (8), the steps of the algorithm will now be described with reference to FIG. 3.

Referring to FIG. 3, for ultrasound machines that generate and store coherent images, as described herein above, the first step, denoted 30, is the generation of a set of images by moving the ultrasound transducer 10 in a circular arc 18 about a center point 14. The next step, denoted 40, is reconstructing the carrier frequency by multiplying each image by a function of the form,

e^{-ik(Xi cos θ+Yj sin θ).} Eq. (9)

This step may not be necessary if the stored data already contains the carrier information within it. It should be noted that Eq. (8) describes data already containing the carrier function. The intent is to render the data in a manner consistent with the expression of Eq. (8). The next step, denoted 50, is multiplying the image pixel at position (xᵢ, yⱼ) by a phase factor,

e^{-ik(Xo cos θ+Yo sin θ).} Eq. (10)

The phase function in Eq. (10) is just the conjugate of the second factor in Eq. (8) for a point target at point (xᵢ, yⱼ).

The next step, denoted 60, is the rotation of all images to align the common x-y coordinate system to gain a more complete picture of the entire pixel range within the target area 12.

The next step, denoted 70, is to sum the value of that pixel for each θ-dependent image as in Eq. (6). After producing a highly resolved image of a single pixel through mathematical imaging using the mathematical process described above, the process is repeated with the next pixel, as indicated by decision diamond 75 and block 80. Thus, step 70 is repeated until all of the pixels have been processed.

The summation of the coherent images from step 70 will generally result in an image for which each pixel has both magnitude and phase components. While such a pixel image can be stored as a coherent-sum image, for viewing, however, the information of interest is merely the magnitude of each pixel. Therefore, it is necessary to strip the phase component from the image prior to submission to a display device. This operation, denoted as step 90, is typically referred to as magnitude detection.

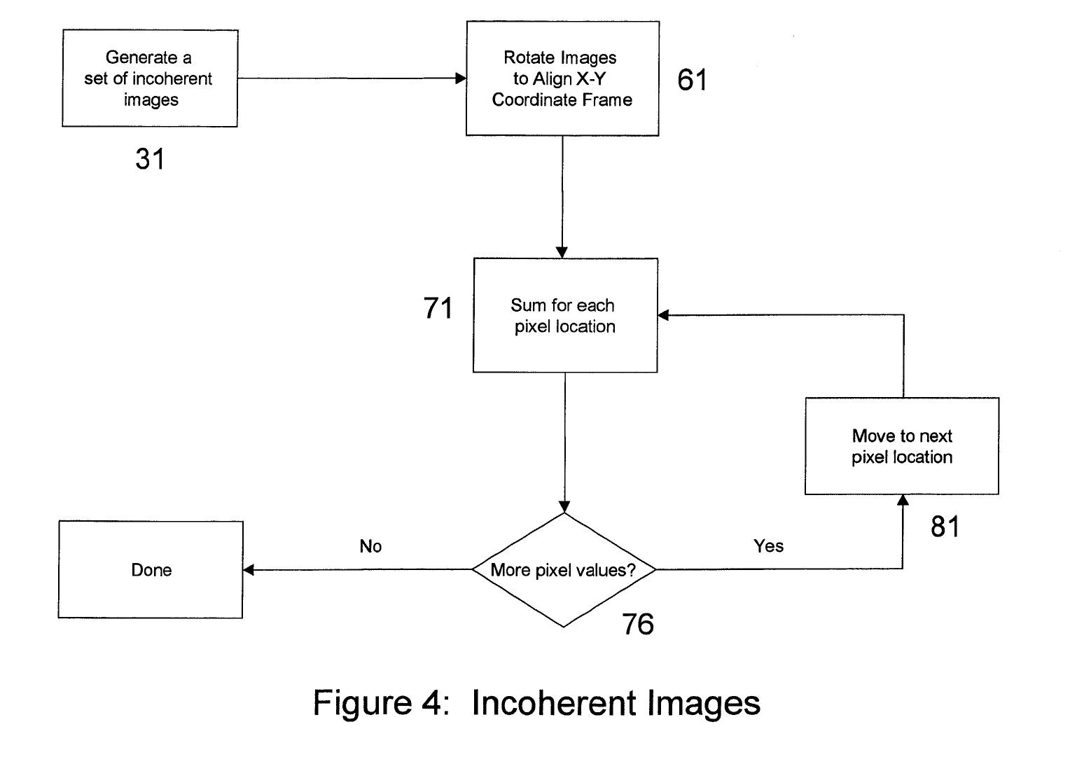

The process is far less complex for ultrasound machines that do not store coherent images. Referring to FIG. 4, for ultrasound machines that do not store coherent images, the first step, denoted 31, is the generation of a set of images by moving the ultrasound transducer 10 in a circular arc 18 about a center point 14.

The next step, here denoted 61, is the rotation of all images to align the common x-y coordinate system to gain a more complete picture of the entire pixel range within the target area 12.

The next step, denoted 71, is to sum the value of that pixel for each □-dependent image as in Eq. (6), above. After producing a highly resolved image of a single pixel through mathematical imaging using the mathematical process described above, the process is repeated with the next pixel, as indicated by decision diamond 76 and block 81. Thus, step 71 is repeated until all of the pixels have been processed—producing a highly resolved, final image.

Additionally, it can be appreciated that this technique can be extended to produce 3-dimensional, highly resolved images if the individual images are taken from adjacent non-planar arcs or from a longitudinal spiral pattern.

As indicated above, one important application of the present invention is in breast cancer detection and treatment. Currently, breast cancer is the number one cause of cancer-related deaths in women. With higher resolution imaging, breast cancer could be detected earlier and more accurately, and diagnoses could be more precise. In comparison to the other techniques described above, ultrasound has the advantage of being a non-ionizing modality that has not been shown to cause cancer, and can be used in real time in combination with fine needle aspiration in biopsy procedures. Ultrasound equipment is relatively inexpensive and portable, particularly as compared with techniques such as computer axial tomography (CAT) and magnetic resonance imaging (MRI). Moreover, patients can be imaged without the painful compression of breast tissue that is currently required in x-ray mammography.

The high-resolution ultrasound imaging herein can overcome a serious disadvantage with current ultrasound techniques—the lack of image resolution. Tests have shown that a substantial enhancement in resolution can be provided with the method of the invention. Moreover, it is projected that the method of the invention will provide an improvement in resolution from 0.5 mm to 0.3 mm at low frequencies and even higher resolution at higher frequencies.

Although the invention has been described above in relation to preferred embodiments thereof, it will be understood by those skilled in the art that variations and modifications can be effected to the preferred embodiments without departing from the scope and spirit of the invention.

We claim:

1. A method of improving the resolution of ultrasound images, said method comprising the steps of:
(a) obtaining an ultrasound image of a target area using an ultrasound imaging apparatus located at a first position outside said target area and at a predetermined distance from a center point within said target area;
(b) moving said ultrasound imaging apparatus along an arc spaced from the center point of the target area by said predetermined distance to a new position and obtaining a further ultrasound image of the target area;
(c) repeating step (b) for a plurality of further positions along said arc until the ultrasound apparatus has been moved up to 360° along said arc;
(d) calculating the phase of sound waves returned from a pixel within said target area to said ultrasound imaging apparatus for all of said positions of said ultrasound imaging apparatus so as to obtain image data for the pixel;
(e) combining the image data for the pixel calculated for all of said positions of said ultrasound imaging apparatus to produce a resolved image of the pixel; and
(f) repeating steps (d) and (e) for each pixel within said target area until all pixels within said target area are resolved.

2. A method according to claim 1, wherein said ultrasound apparatus is moved between 2° and 5° around said arc between each of said further positions.

3. A method according to claim 1, wherein the ultrasound images comprise incoherent images, and wherein said incoherent images are combined, using a synthetic aperture algorithm, into an improved-resolution incoherent image.

4. A method according to claim 1, wherein the ultrasound images from the ultrasound apparatus are coherent images, and wherein the coherent images are combined into an improved-resolution coherent image.
